(19) United States
(12) Patent Application Publication
(10) Pub. No.: US 2019/0192662 A1
(43) Pub. Date: Jun. 27, 2019

(54) TRANSDERMAL PHARMACEUTICAL COMPOSITIONS WITH REDUCED SKIN IRRITATION

(71) Applicant: NOVEN PHARMACEUTICALS, INC., Miami, FL (US)

(72) Inventors: Swetha INTURI, Miami, FL (US); Jeffrey KLEIN, Miami, FL (US)

(73) Assignee: NOVEN PHARMACEUTICALS, INC., Miami, FL (US)

(21) Appl. No.: 16/224,487

(22) Filed: Dec. 18, 2018

Related U.S. Application Data
(60) Provisional application No. 62/609,955, filed on Dec. 22, 2017.

(51) Int. Cl. A61K 47/22; A61K 9/00; A61K 31/4525

(52) U.S. Cl. CPC A61K 47/22; A61K 31/4525; A61K 9/0014

(57) ABSTRACT

Described are transdermal and transmucosal pharmaceutical compositions exhibiting reduced skin or mucosa irritation potential comprising a phytoestrogen and an antioxidant, useful for reducing the skin or mucosa irritation associated with the use of transdermal or transmucosal drug delivery compositions. Methods of making and using the compositions also are described.

### CROSS-REFERENCE TO RELATED APPLICATIONS

This application claims the priority benefits under 35 USC § 119 to U.S. provisional application 62/609,955, filed Dec. 22, 2017, the entire contents of which are incorporated herein by reference in their entirety.

### FIELD OF THE INVENTION

This invention relates generally to pharmaceutical compositions for transdermal or transmucosal drug delivery, and more particularly to transdermal or transmucosal pharmaceutical compositions that exhibit reduced skin irritation. Methods of making and using the compositions also are described.

### BACKGROUND

The use of transdermal or transmucosal pharmaceutical compositions to administer an active agent through the skin or mucosa of a subject is well known. Non-occlusive transdermal or transmucosal compositions typically incorporate the active agent into a carrier composition with volatile components that evaporate after application, such as liquid, gel, and emulsion compositions, while occlusive transdermal or transmucosal compositions typically incorporate the active agent into a carrier composition with at least some non-volatile components that do not evaporate after application, such as film-forming compositions or polymeric and/or pressure-sensitive adhesive compositions.

Many factors influence the design and performance of transdermal and transmucosal pharmaceutical compositions, such as the individual drugs themselves, the physical/chemical characteristics of the composition's components and the performance/behavior relative to other components once combined, external/environmental conditions during manufacturing and storage thereafter, the properties of the topical site of application, the desired rate of drug delivery and onset, the drug delivery profile, and the intended duration of delivery.

Transdermal and transmucosal pharmaceutical compositions may be associated with skin or mucosal irritation. The nature and extent of skin or mucosal irritation may vary with the drug and other components formulated in the compositions. For occlusive compositions, the duration of use of the system (e.g., how long a given system is left in place) also may impact the nature and extent of skin irritation. Even when a transdermal or transmucosal pharmaceutical composition exhibits a good therapeutic effect, skin or mucosal irritation may undermine patient compliance and/or discourage the prescription or use of a given transdermal or transmucosal pharmaceutical composition.

While agents for reducing skin irritation associated with the use of transdermal or transmucosal pharmaceutical compositions are known, such agents may impact the performance of transdermal or transmucosal pharmaceutical compositions, such as by reacting with the drug or other components formulated in the composition, impacting the drug delivery profile, reducing drug delivery to an ineffective level, and/or impacting the physical properties of the composition (e.g., the adhesion and wear properties of an occlusive system). Thus, there remains a need for transdermal and transmucosal pharmaceutical compositions that exhibit reduced skin irritation.

### SUMMARY OF THE INVENTION

Described are transdermal and transmucosal pharmaceutical compositions that exhibit reduced skin or mucosal irritation, comprising a phytoestrogen and an antioxidant.

Some embodiments relate to transdermal or transmucosal pharmaceutical compositions comprising a drug to be delivered transdermally or transmucosally, and a phytoestrogen and an antioxidant in amounts effective to reduce the skin or mucosa irritation potential of the composition. The phytoestrogen may comprise one or more selected from daidzein, genistein, formononetin, glycitein, biochanin A, coumestrol, trifoliol, pinoresinol, lariciresinol, secoisolariciresinol, matairesinol, hydroxymatairesinol, syringaresinol, sesamin, 8-prenylnaringenin/hopein, apigenin, resveratrol, and ellagitannins. The antioxidant may comprise one or more selected from epigallocatechin gallate, quercetin, α-bisobolol, rosamarinic acid, puerarin, hamamelitanin, aluminum acetate, curcumin, glycyrrhizin, retinol acetate, l-ascorbyl palmitate, tocopherol, calcipotriol, tacalcitol, resveratrol, silibinin, ginsenoside, squalene, aluminum hydroxide, titanium oxide, ethylene diamine tetraacetic acid (EDTA), pantethine, and tranexamic acid. In specific embodiments, the phytoestrogen comprises one or more selected from daidzein and genistein and/or the antioxidant comprises one or more selected from epigallocatechin gallate and quercetin.

In some embodiments, the phytoestrogen and antioxidant exhibit a skin or mucosal irritation reducing effect that is greater than that of the phytoestrogen or antioxidant alone. In some embodiments, neither the phytoestrogen alone nor the antioxidant alone exhibit a skin or mucosal irritation reducing effect.

In some embodiments, the composition is a non-occlusive or film-forming composition and the phytoestrogen is present in an amount of from about 0.1 to about 20 mg/mL based on the total volume of the composition, and/or the antioxidant is present in an amount of from about 0.05 to about 10 mg/mL based on the total volume of the composition.

In some embodiments, the composition is a transdermal or transmucosal drug delivery system in the form of a flexible finite system for topical application. In some embodiments, the composition comprises amounts of the phytoestrogen and antioxidant effective to reduce skin or mucosal irritation associated with use of the system over an application period of at least one day, at least three days, or at least seven days. In some embodiments, the phytoestrogen and antioxidant are present in a skin- or mucosa-contacting layer of the system. In some embodiments, the phytoestrogen and antioxidant are present in a drug-containing polymer matrix layer of the system.

Some embodiments relate to methods of reducing the skin or mucosal irritating effects of a transdermal or transmucosal pharmaceutical composition, comprising formulating a transdermal or transmucosal pharmaceutical composition with a phytoestrogen and an antioxidant in amounts effective to reduce the skin or mucosa irritation potential of the composition.

Some embodiments relate to methods of reducing the skin or mucosal irritating effects of transdermal drug delivery, comprising administering to a subject in need of transdermal or transmucosal drug delivery a transdermal or transmucosal pharmaceutical composition comprising a phytoestrogen and an antioxidant in amounts effective to reduce the skin or mucosa irritation potential of the composition.

Some embodiments relate to uses of a phytoestrogen and an antioxidant in the preparation of a medicament for the transdermal or transmucosal delivery of a drug, wherein the medicament is in the form of a transdermal or transmucosal pharmaceutical composition comprising the phytoestrogen, antioxidant, and drug to be delivered transdermally or transmucosally, wherein the phytoestrogen and antioxidant are present in amounts effective to reduce the skin or mucosa irritation potential of the composition.

Some embodiments relate to transdermal or transmucosal pharmaceutical compositions comprising a drug to be delivered transdermally or transmucosally, and a phytoestrogen and an antioxidant in amounts effective to reduce the skin or mucosa irritation potential of the composition, for reducing skin or mucosal irritation associated with transdermal or transmucosal delivery of the drug.

### BRIEF DESCRIPTION OF THE DRAWINGS

FIG. 1A-C show the drug flux through human skin of paroxetine from different formulations described in the examples. Each of FIGS. 1A-C represents the drug flux through a given skin sample.

### DETAILED DESCRIPTION

Described are transdermal and transmucosal pharmaceutical compositions that exhibit reduced skin or mucosal irritation associated with the use of transdermal or transmucosal pharmaceutical compositions.

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth, and is intended to refer to values substantially around the quoted value while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein means that the described composition (e.g., polymer matrix, etc.) comprises less than about 5%, less than about 3%, or less than about 1% by weight, based on the total weight of the composition at issue, of the excluded component(s).

As used herein, “subject” denotes any mammal, including humans. For example, a subject may be suffering from or at risk of developing a condition that can be diagnosed, treated or prevented with a drug as described herein, or may be taking a drug for other purposes.

As used herein “drug” denotes any pharmaceutically or physiologically active agent, whether or not approved for use to prevent or treat a specific disease, disorder, or condition, including any agent having diagnostic, prophylactic, therapeutic, pharmacological, physiological, or cosmetic effect, including diagnostic agents, therapeutic agents, insect repellents, pesticides, sun screens, cosmetic agents, etc.

As used herein, “transdermal pharmaceutical composition” refers to a pharmaceutical composition formulated to deliver drug through the skin of a subject, and includes non-occlusive and occlusive transdermal pharmaceutical compositions. As used herein, a “non-occlusive transdermal pharmaceutical composition” refers to a composition that does not substantially occlude the surface of the skin to which it is applied. In a typical non-occlusive transdermal composition, the drug is incorporated into a carrier composition with volatile components that evaporate after application, such as liquid, gel, ointment, and emulsion compositions. As used herein, an “occlusive transdermal pharmaceutical compositions” refers to a composition that substantially occludes the surface of the skin to which it is applied, such as a film-forming composition or transdermal patch-type system (also referred to herein as a “transdermal drug delivery system”). As used herein, “transdermal” is separate and distinct from “transmucosal”, and refers to delivery through skin in particular, while “transmucosal” refers to delivery through mucosa.

As used herein, “transmucosal pharmaceutical composition” refers to a pharmaceutical composition formulated to deliver drug through the mucosa of a subject, and includes non-occlusive and occlusive transdermal pharmaceutical compositions. As used herein, a “non-occlusive transmucosal pharmaceutical composition” refers to a composition that does not substantially occlude the surface of the mucosa to which it is applied. In a typical non-occlusive transmucosal composition, the drug is incorporated into a carrier composition with volatile components that evaporate after application, such as liquid, gel, ointment, and emulsion compositions. As used herein, an “occlusive transmucosal pharmaceutical compositions” refers to a composition that substantially occludes the surface of the mucosa to which it is applied, such as a film-forming composition or mucosal patch-type system (also referred to herein as a “transmucosal drug delivery system”).

As used herein, “transdermal drug delivery system” and “transmucosal drug delivery system” refer to compositions in a “flexible, finite form,” which refers to a substantially solid form capable of conforming to a surface with which it comes into contact, and capable of maintaining contact so as to facilitate topical application. Such systems in general are known in the art and commercially available, such as transdermal and transmucosal drug delivery patches.

As used herein, the terms “topical” and “topically” mean application to a skin or mucosal surface of a mammal, while the terms “transdermal” and “transmucosal” connote passage through the skin or mucosa, respectively, with mucosa including oral, buccal, nasal, rectal and vaginal mucosa, into systemic circulation. Thus, the systems described herein may be applied topically to a subject to achieve transdermal or transmucosal delivery of a drug.

As used herein, the phrases “therapeutically effective amount” and “therapeutic level” mean that drug dosage or plasma concentration in a subject, respectively, that provides the specific effect for which the drug is administered in a subject receiving the treatment. It is emphasized that a therapeutically effective amount or therapeutic level of a drug will not always be effective in achieving the intended effect, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein “flux” is defined as the percutaneous absorption of drugs through the skin, and is described by Fick's first law of diffusion:

J=−D (dCₘ/dx),

where J is the flux in g/cm²/sec, D is the diffusion coefficient of the drug through the skin in cm²/sec and dCₘ/dx is the concentration gradient of the active agent across the skin or mucosa.

### Reduced Irritation Compositions

Described herein are transdermal and transmucosal pharmaceutical compositions comprising agents that reduce skin or mucosal irritation associated with the use of transdermal and transmucosal pharmaceutical compositions, referred to herein as “skin irritation reducing agents”. The agents may include a phytoestrogen and an antioxidant. It has been surprisingly discovered that such agents can reduce the skin or mucosa irritation potential of a transdermal or transmucosal pharmaceutical composition while still achieving sufficient flux of drug through the skin or mucosa to achieve the intended effect, e.g., without unduly inhibiting the efficacy of the compositions.

The skin or mucosa irritation potential of a transdermal or transmucosal pharmaceutical composition can be assessed by methods known in the art, such as by in vitro testing or animal testing. For example, in vitro testing can be conducted using an Epiderm™ skin model (MatTek Corporation, Ashland, Mass., USA) and an MTT assay, as illustrated in the examples. Animal testing can be conducted using any suitable animal model, such as a rabbit skin irritation test with scoring in accordance with the Primary Irritation Index PII (see, e.g., ASTM F719-81).

In specific embodiments, while neither the phytoestrogen nor antioxidant alone would be effective to reduce the skin or mucosa irritation potential of the composition, the combination of the phytoestrogen and antioxidant is effective to reduce the skin or mucosa irritation potential of the composition. In other specific embodiments, while neither the phytoestrogen nor antioxidant alone would be effective to reduce the skin or mucosa irritation potential of the composition in the amounts used, the combination of the phytoestrogen and antioxidant in the amounts used is effective to reduce the skin or mucosa irritation potential of the composition.

Phytoestrogens are structurally similar to human estrogens, and are found in plants or derived from plant precursors. Examples of phytoestrogens suitable for use as a skin irritation reducing agent include phytoestrogen isoflavones, such as daidzein, genistein, formononetin, glycitein, and biochanin A, lignans, as well as other types of phytoestrogens including coumestans (such as coumestrol and trifoliol), lignans (such as pinoresinol, lariciresinol, secoisolariciresinol, matairesinol, hydroxymatairesinol, syringaresinol and sesamin), flavonoids (such as 8-prenylnaringenin/hopein, apigenin), stilbenes (such as resveratrol) and ellagitannins.

In some embodiments, the amount of phytoestrogen used is not effective for systemic effect. That is, while the amount used is effective (alone or in combination with an antioxidant) to reduce the skin or mucosa irritation potential of the composition at the site of application of the composition, it does not exhibit a systemic physiological effect on the subject. In some embodiments, the phytoestrogen does not flux through the skin, or does not flux through the skin to a detectable extent, or does not flux through the skin to a physiologically meaningful extent, or does not flux through the skin to an extent sufficient to exert a local in vivo effect, or does not flux through the skin to an extent sufficient to exert a systemic in vivo effect. In some embodiments, the phytoestrogen does not flux through the mucosa, or does not flux through the mucosa to a detectable extent, or does not flux through the mucosa to a physiologically meaningful extent, or does not flux through the mucosa to an extent sufficient to exert a local in vivo effect, or does not flux through the mucosa to an extent sufficient to exert a systemic in vivo effect. In some embodiments of non-occlusive or film-forming compositions, the phytoestrogen is used in an amount of from about 0.1 to about 20 mg/mL, including from about 0.2 to about 10 mg/mL, based on the total volume of the transdermal or transmucosal pharmaceutical composition.

Examples of antioxidants suitable for use as a skin irritation reducing agent include epigallocatechin gallate, quercetin, α-bisobolol, rosamarinic acid, puerarin, hamamelitanin, curcumin, glycyrrhizin, retinol acetate, l-ascorbyl palmitate, tocopherol, calcipotriol, tacalcitol, resveratrol, silibinin, ginsenoside, squalene, ethylene diamine tetraacetic acid (EDTA), pantethine, trans-ferulic acid, hesperidin, and allantoin.

In some embodiments, the amount of antioxidant used is not effective for systemic effect. That is, while the amount used is effective (alone or in combination with a phytoestrogen) to reduce the skin or mucosa irritation potential of the composition at the site of application of the composition, it does not exhibit a systemic physiological effect on the subject. In some embodiments of non-occlusive or film-forming compositions, the antioxidant is used in an amount of from about 0.05 to about 10 mg/mL, including from about 0.05 to about 1.0 mg/mL, and from about 0.1 to about 0.5 mg/mL, based on the total volume of the composition.

Any one or more phytoestrogen(s) can be used with any one or more antioxidant(s). If a combination of phytoestrogen(s) and/or antioxidant(s) is used, the total amount of phytoestrogen(s) and/or antioxidant(s) typically will be within the ranges set forth above.

In specific embodiments, a composition exhibiting reduced skin or mucosal irritation comprises a phytoestrogen selected from daidzein and genistein (or a combination thereof) and an antioxidant selected from epigallocatechin gallate and quercetin (or a combination thereof).

### Transdermal and Transmucosal Pharmaceutical Compositions

As noted above, transdermal and transmucosal pharmaceutical compositions that can be formulated as described herein with reduced skin or mucosa irritation potential include non-occlusive transdermal and transmucosal pharmaceutical compositions and occlusive transdermal and transmucosal pharmaceutical compositions. Examples of non-occlusive transdermal and transmucosal pharmaceutical compositions include liquid, gel, ointment, cream, and emulsion compositions. Examples of occlusive transdermal and transmucosal pharmaceutical compositions include film-forming compositions that may be applied as a liquid, gel, ointment, cream, or emulsion and form a film on the skin or mucosal surface upon evaporation of volatile components, and patch-type systems which are discussed in more detail below. For any transdermal or transmucosal pharmaceutical composition, the drug may be formulated in any suitable form, including being dissolved, dispersed or suspended in a carrier composition. In embodiments where the drug is in solid form, the drug may be in any solid form, including any crystalline or amorphous form, and/or provided as particles (e.g., drug-encapsulated particles, coated drug particle, drug nanoparticles, etc.).

Typically, the compositions are formulated to provide a diagnostically or therapeutically effective amount of drug, e.g., an amount of drug effective to exert the intended effect, based on the amount of composition to be applied. The amount of drug formulated in the composition may depend on the composition being prepared, the particular drug being formulated, the desired effect, and the duration for which the composition is to provide therapy. For most drugs, the passage of the drugs through the skin or mucosa will be the rate-limiting step in delivery. Thus, the minimum amount of drug in the composition may be selected based on the amount of drug which passes through the skin or mucosa from the composition and the in vivo drug level needed to exert the intended effect. The maximum amount of drug in a composition may be limited by the solubility of the drug in the composition or other chemical or physical properties and/or by the amount of drug to be delivered. The amount of drug in a composition can vary from as little as 0.001% or less to 95% or more.

In some embodiments, the composition may include a skin or mucosa penetration enhancer. The term “enhancer” as used herein refers to substances used to increase permeability and/or accelerate the delivery of an active agent through the skin or mucosa. Enhancers suitable for use in transdermal or transmucosal pharmaceutical compositions are known in the art, and may depend on the drug being formulated and the specific type of composition (e.g., liquid, gel, film-forming, patch, etc.). Typically, an enhancer (or combination thereof) may be used in a transdermal or transmucosal pharmaceutical composition in an amount of up to about 50% by weight of the composition, or in any amount sufficient to yield a permeation-enhancing effect without rendering the composition as a whole unsuitable for its intended purpose.

In some embodiments, the composition may include one or more other pharmaceutically acceptable additives or excipients frequently used in such compositions. Such additives and excipients include diluents, stabilizers, buffering agents, biocides, humectants, anti-irritants, antioxidants, preservatives, flavoring agents, colorants, pigments and the like. Such substances can be present in any amount sufficient to impart the desired properties to the composition without rendering the composition as a whole unsuitable for its intended purpose. Typically, such additives or excipients are used in amounts up to 25% of the composition.

### Transdermal and Transmucosal Drug Delivery Systems

In specific embodiments, the transdermal or transmucosal pharmaceutical composition is a transdermal or transmucosal drug delivery system. The transdermal or transmucosal drug delivery systems may be of any type known in the art, including drug-in-adhesive matrix-type or reservoir-type systems, and may comprise one or more drug-containing layers, reservoir layers, pressure-sensitive adhesive or bioadhesive layers (whether or not containing drug), overlay layers, etc., as well as a backing and removable release liner. When present, the skin/mucosal irritation-reducing agents as described herein may be present in any layer, but typically are present in a skin- or mucosal-contacting layer, such as a skin- or mucosal-contacting layer of a drug-in-adhesive matrix-type system or a skin-contacting layer of a reservoir-type system.

A drug-in-adhesive matrix-type system typically includes a drug-containing polymer matrix, which refers to a polymer composition which contains one or more drugs and one or more polymers, such as one or more pressure-sensitive adhesive and/or bioadhesive/mucoadhesive polymers. As used herein, “active surface area” means the surface area of the drug-containing polymer matrix of the transdermal or transmucosal drug delivery system. A polymer is an “adhesive” or “bioadhesive/mucoadhesive” if it has the properties of adhesiveness per se. Other polymers can function as an adhesive or bioadhesive/mucoadheisve by the addition of tackifiers, plasticizers, crosslinking agents or other excipients. Thus, in some embodiments, the drug-containing polymer matrix optionally comprises tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, a polymer is a pressure-sensitive adhesive polymer if it has the properties of a pressure-sensitive adhesive per se. Other polymers may function as a pressure-sensitive adhesive by admixture with tackifiers, plasticizers or other additives. The term pressure-sensitive adhesive also includes mixtures of different polymers.

In some embodiments, the polymer matrix is a pressure-sensitive adhesive at room temperature and exhibits desirable physical properties, such as good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

As used herein, the terms “bioadhesive” and “mucoadhesive” refer to materials that adhere to mucosal surfaces, e.g., that adhere to surfaces upon wetting or hydration. As noted above, a polymer is a bioadhesive/mucoadhesive polymer if it has the properties of a bioadhesive/mucoadhesive per se. Other polymers may function as bioadhesives/mucoadhesives by admixture with tackifiers, plasticizers or other additives. The term bioadhesive/mucoadhesive also includes mixtures of different polymers.

In some embodiments, the systems are “monolithic” or “monolayer” systems, such that the drug-containing polymer matrix layer is the only polymeric layer present other than the backing layer and the release liner, if present. In such embodiments, the polymer matrix functions as both the drug carrier and the means of affixing the system to the skin or mucosa. In such embodiments, the skin irritation-reducing agents as described herein are present in the drug-containing polymer matrix layer, which also is the skin- or mucosa-contacting layer.

In other embodiments, the systems are multi-layer systems that include one or more drug-containing polymer matrix layers, or one or more non-drug containing polymer matrix layers in addition to one or more drug-containing polymer matrix layers. In such embodiments, the one or more additional layers may impart desired adhesion, wear, or pharmacokinetic properties, for example. In such embodiments, the skin irritation-reducing agents as described herein may be present in any layer, but typically are present in the skin- or mucosa-contacting layer, e.g., the layer adjacent the release liner (if present) prior to its removal.

In other embodiments, the systems are reservoir systems that include a reservoir and an adhesive layer for adhering the system to the skin or mucosa. In such embodiments, the skin or mucosal irritation-reducing agents as described herein may be present in any portion of the system, but typically are present in the skin- or mucosal-contacting layer, e.g., the adhesive layer adjacent the release liner (if present) prior to its removal, or in the reservoir.

Suitable polymers, adhesive, and excipients for use in transdermal and transmucosal drug delivery systems are known in the art. For example, suitable pressure-sensitive adhesives include solvent-based, hot melt and grafted adhesives, which may be used alone or in combinations, mixtures or blends. Examples include acrylic-based polymer(s), silicone-based polymer(s), rubbers, gums, polyisobutylenes, polyvinylethers, polyurethanes, styrene block copolymers, styrene/butadiene polymers, polyether block amide copolymers, ethylene/vinyl acetate copolymers, and vinyl acetate-based adhesives. Suitable bioadhesive/mucoadhesive polymers include carbomers, carboxyethylene polymers, and/or polyacrylic acids, poly(2-hydroxyethyl methacrylate) (PHEMA), poly(methacrylic acid), polyethylene oxides, poloxamers, cellulose derivatives such as hydroxypropylmethylcellulose (HPMC), ethylhydroxyethylcellulose (EHEC), carboxymethylcellulose (CMC), salts of carboxymethylcellulose, hydroxypropylcellulose (HPC), hydroxyethylcellulose (HEC), hydroxyethylmethylcellulose, arabic gum, shiraz gum, karaya gum, xanthan gum, guar gum, gellan gum, alginates (e.g., sodium alginate), hyaluronates (e.g., hyaluronic acid, sodium hyaluronate), polyvinylpyrrolidones, chitosans (e.g., chitosan, thiolated chitosan), pectin, gelatin, polyvinyl alcohols, and veegum. U.S. Pat. No. 6,562,363 describes mucoadhesive polymers (also referred to as “bioadhesive polymers) that are useful in the transmucosal compositions described herein.

For most drugs, the passage of the drugs through the skin or mucosa will be the rate-limiting step in delivery. Thus, the minimum amount of drug in the system is selected based on the amount of drug which passes through the skin or mucsoa from the composition during the duration which the device is to provide therapy. Normally, the amount of drug in a system can vary from as little as 0.1% or less to about 50% or more of the layer in which it is incorporated. In a reservoir-type system, the reservoir composition may be comprised of substantially only drug (e.g., if the drug is a liquid), or of a highly concentrated (e.g., saturated) composition of drug in a liquid, gel, semi-solid or solid matrix.

A system may include a skin or mucosa penetration enhancer as discussed above in one or more of the layers. An enhancer may be formulated in any layer(s) of the system, but typically is formulated in a drug-containing layer and/or a skin-contacting layer. Typically, an enhancer (or combination thereof) may be used in a transdermal or transmucosal drug delivery system in an amount of up to about 30% by weight, based on the dry weight of the layer in which it is formulated, such as from about 0.1% to about 15%, or in any amount sufficient to yield a permeation-enhancing effect.

A system may include one or more other pharmaceutically acceptable additives or excipients frequently used in such systems, which may be present in any one or more layers of the system. Such additives include tackifying agents such as aliphatic hydrocarbons, mixed aliphatic and aromatic hydrocarbons, aromatic hydrocarbons, substituted aromatic hydrocarbons, hydrogenated esters, polyterpenes, silicone fluid, mineral oil and hydrogenated wood rosins. Additional additives include binders such as lecithin which “bind” the other ingredients, or rheological agents (thickeners) containing silicone such as fumed silica, reagent grade sand, precipitated silica, amorphous silica, colloidal silicon dioxide, fused silica, silica gel, quartz and particulate siliceous materials commercially available as Syloid®, Cabosil®, Aerosil®, and Whitelite®, for purposes of enhancing the uniform consistency or continuous phase of the composition or coating. Other additives and excipients include diluents, stabilizers, fillers, clays, buffering agents, biocides, humectants, anti-irritants, antioxidants, preservatives, plasticizing agents, cross-linking agents, flavoring agents, colorants, pigments and the like. Such substances can be present in any one or more layers, and can be present in any amount sufficient to impart the desired properties to the layer at issue or system as a whole. Typically, such additives or excipients are used in amounts up to 25% based on the dry weight of the layer(s) in which they are formulated, such as from about 0.1% to about 10%.

As noted above, transdermal drug delivery systems typically include a backing and a removable release liner to protect and/or anchor the system or its components during manufacturing as described herein, or thereafter, and to enable handling and transportation. Suitable materials for use as backings and release liners are known in the art. Transmucosal drug delivery systems may or may not include a backing, and may or may not include a removable release liner

An exemplary general method of preparing a transdermal drug delivery system is as follows:
Appropriate amounts of polymer(s), solvent(s), enhancer(s), and organic solvent(s) (for example toluene) are combined and thoroughly mixed together in a vessel.
Drug and skin irritation-reducing agents are added to the mixture and agitation is carried out until the drug is uniformly mixed in.
The formulation is then transferred to a coating operation where it is coated onto a protective release liner at a controlled specified thickness. The coated product is then passed through an oven in order to drive off all volatile processing solvents.
The dried product on the release liner is then joined to the backing material and wound into rolls for storage.
Thereafter, desired size and shape delivery systems are prepared by die-cutting or the like, from the rolled laminate and then packaged.

Transmucosal delivery systems can be made by analogous methods, or by any other methods known in the art. In some embodiments, a method of manufacture comprises blending (mixing) the bioadhesive polymer(s) and, as needed, other excipients, in the presence of an appropriate solvent, and casting the wet blend onto a solid support (such as a release liner or backing), or dispending into a mold. followed by evaporation of the solvent(s) at appropriate drying conditions. Optionally, a backing layer (if being used, and not already used as a support) can be applied (e.g., laminated) to the dried mucoadhesive layer.

### Therapeutic Methods

As noted above, the compositions described herein can be used to transdermally or transmucosally administer any drug, for any desired effect, including diagnostic, prophylactic, therapeutic, or cosmetic effect. In such methods, a composition as described herein is applied to the skin or mucosa of the subject. In some embodiments using a transdermal or transmucosal drug delivery system, a system is applied for about one day. In other embodiments using a transdermal or transmucosal drug delivery system, a system is applied for more than one day, such as for two days, three days, four days, five days, six days, seven days, or longer.

In specific embodiments, the use of a composition as described herein, comprising skin irritation reducing agents as described herein, reduces the skin or mucosal irritation that otherwise would be associated with the transdermal or transmucosal delivery of the drug from a comparable composition formulated without the skin irritation reducing agents, e.g., formulated without the steroid(s) and antioxidant(s). That is, the skin irritation reducing agents are effective to reduce the skin or mucosa irritation potential of the composition. Thus, in some embodiments, compositions as described herein, comprising skin irritation reducing agents as described herein, can be used for a longer wear period without being associated with unacceptable skin or mucosal irritation.

Also provided herein are methods of reducing the skin or mucosal irritating effects of a transdermal or transmucosal pharmaceutical composition, comprising formulating a transdermal or transmucosal pharmaceutical composition with skin irritation reducing agents including a phytoestrogen and an antioxidant effective to reduce the skin or mucosa irritation potential of the composition, and methods of reducing the skin or mucosal irritating effects of transdermal or transmucosal drug delivery, comprising administering to a subject in need of transdermal or transmucosal drug delivery a transdermal or transmucosal pharmaceutical composition comprising a phytoestrogen and an antioxidant effective to reduce the skin or mucosa irritation potential of the composition.

The following specific examples are included as illustrative of the compositions described herein. These examples are in no way intended to limit the scope of the invention. Other aspects of the invention will be apparent to those skilled in the art to which the invention pertains.

### EXAMPLES

In vitro testing can be conducted using an Epiderm™ skin model (MatTek Corporation, Ashland, Mass., USA) and an MTT assay.

As described by MatTek Corporation, Epiderm™ is a Reconstructed Human Epidermis (RHE) product that is a highly differentiated three-dimensional tissue model consisting of normal, human-derived epidermal keratinocytes (NHEK), that exhibits human epidermal tissue structure and cellular morphology, having a three-dimensional structure consisting of organized and proliferative basal cells, spinous and granular layers, and cornified epidermal layers that are mitotically and metabolically active.

The MTT (3-[4,5-dimethylthiazol-2-yl]-2,5-diphenyltetrazolium bromide) conversion assay measures the NAD(P)H-dependent microsomal enzyme reduction of MTT (and to a lesser extent, the succinate dehydrogenase reduction of MTT) to a blue formazan precipitate, and can be used to assess cellular metabolism after exposure to a test composition.

Paroxetine was used as a representative drug associated with skin irritation when delivered transdermally. Paroxetine compositions were prepared by mixing saturated solutions of paroxetine in mineral oil with mineral oil at a ratio of 1:9 or 1:4 to arrive at 1/10^{th} (1:10) or ⅕^{th} (1:5) saturated compositions, which were formulated with or without phytoestrogen and/or antioxidant, and assessed using the Epiderm™ skin model and an MTT conversion assay. Skin viability was assessed after 24 hours exposure to the test compositions. The test compositions and results are reported in the following tables.

| Test Compositions | Relative Viability |
|---|---|
| (daidzein and/or epigallocatechin gallate) | (% of control) |
| paroxetine in mineral oil (1:10) | 6.4 |
| paroxetine in mineral oil (1:10) + | 8.4 |
| daidzein (0.4 mg/mL) | |
| paroxetine in mineral oil (1:10) + | 0 |
| epigallocatechin gallate (0.4 mg/mL) | |
| paroxetine in mineral oil (1:10) + | 27 |
| daidzein (0.4 mg/mL) + | |
| epigallocatechin gallate (0.4 mg/mL) | |

The results show that exposure to the paroxetine solution reduced skin viability, and that daidzein or epigallocatechin gallate alone did not significantly impact skin viability. In contrast, the combination of daidzein and epigallocatechin gallate resulted in a relative viability of 27%, indicating a significant skin irritation-reducing effect.

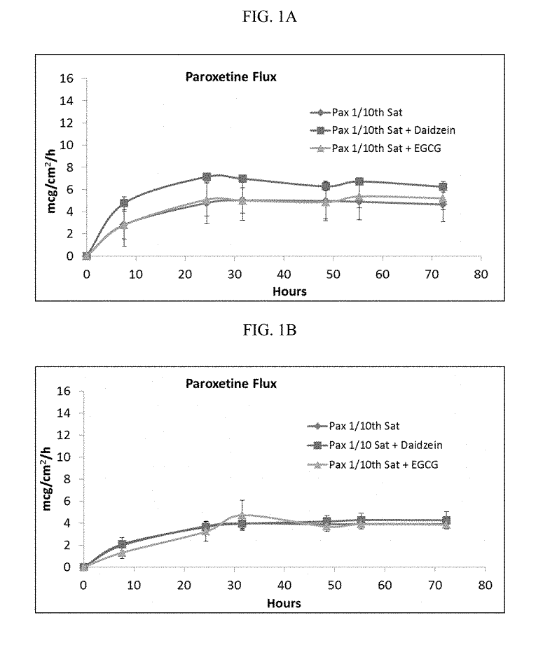

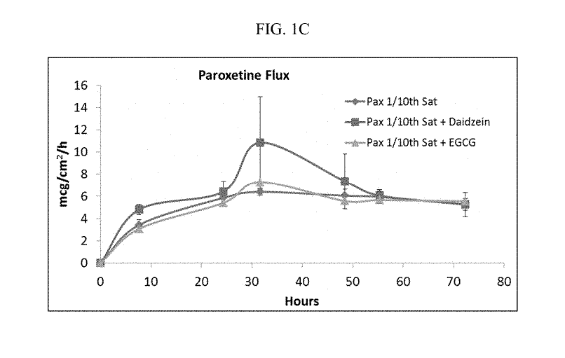

Human skin flux studies confirmed that the ability of paroxetine to be administered transdermally was not reduced by the presence of either daidzein or epigallocatechin gallate in the mineral oil mixture (data not shown) (FIGS. 1A-1C). The flux results also support the conclusion that the anti-irritant effect of the phytoestrogen and antioxidant is not due to reduced drug delivery.

| Test Composition | Relative Viability |
|---|---|
| (genistein and/or quercetin) | (% of control) |
| paroxetine in mineral oil (1:5) | 4.8 |
| paroxetine in mineral oil (1:5) + | 5.3 |
| genistein (0.4 mg/mL) | |
| paroxetine in mineral oil (1:5) + | 22.4 |
| quercetin (0.2 mg/mL) | |
| paroxetine in mineral oil (1:5) + | 25.5 |
| genistein (0.4 mg/mL) + | |
| quercetin (0.2 mg/mL) | |

The results show that exposure to the paroxetine solution reduced skin viability, and that genistein alone did not significantly impact skin viability. In contrast, the combination of genistein and quercetin resulted in a relative viability of 25.5%, indicating a strong skin irritation-reducing effect.

The results indicate that different combinations of a phytoestrogen and an antioxidant are effective at reducing skin irritation associated with transdermal administration of paroxetine. Furthermore, the flux study results show that the paroxetine retained its ability to pass through the skin when formulated with the phytoestrogen and/or antioxidant.

What is claimed is:

1. A transdermal or transmucosal pharmaceutical composition comprising a drug to be delivered transdermally or transmucosally, and a phytoestrogen and an antioxidant in amounts effective to reduce the skin or mucosa irritation potential of the composition.

2. The composition of claim 1, wherein the phytoestrogen comprises one or more selected from daidzein, genistein, formononetin, glycitein, biochanin A, coumestrol, trifoliol, pinoresinol, lariciresinol, secoisolariciresinol, matairesinol, hydroxymatairesinol, syringaresinol, sesamin, 8-prenylnaringenin/hopein, apigenin, resveratrol, and ellagitannins.

3. The composition of claim 1, wherein the phytoestrogen comprises one or more selected from daidzein and genistein.

4. The composition of claim 1, wherein the antioxidant comprises one or more selected from epigallocatechin gallate, quercetin, α-bisobolol, rosamarinic acid, puerarin, hamamelitanin, aluminum acetate, curcumin, glycyrrhizin, retinol acetate, l-ascorbyl palmitate, tocopherol, calcipotriol, tacalcitol, resveratrol, silibinin, ginsenoside, squalene, aluminum hydroxide, titanium oxide, ethylene diamine tetraacetic acid (EDTA), pantethine, and tranexamic acid.

5. The composition of claim 1, wherein the antioxidant comprises one or more selected from epigallocatechin gallate and quercetin.

6. The composition of claim 5, wherein the phytoestrogen comprises one or more selected from daidzein and genistein.

7. The composition of claim 1, wherein the phytoestrogen comprises daidzein and the antioxidant comprises epigallocatechin gallate.

8. The composition of claim 1, wherein the phytoestrogen comprises genistein and the antioxidant comprises quercetin.

9. The composition of claim 1, wherein the phytoestrogen and antioxidant exhibit a skin or mucosa irritation reducing effect that is greater than that of the phytoestrogen or antioxidant alone.

10. The composition of claim 1, wherein neither the phytoestrogen alone nor the antioxidant alone exhibit a skin or mucosa irritation reducing effect.

11. The composition of claim 1, wherein the composition is a non-occlusive or film-forming composition and the phytoestrogen is present in an amount of from about 0.1 to about 20 mg/mL based on the total volume of the composition.

12. The composition of claim 1, wherein the composition is a non-occlusive or film-forming composition and the antioxidant is present in an amount of from about 0.05 to about 10 mg/mL based on the total volume of the composition.

13. The composition of claim 1, wherein the composition is a transdermal drug delivery system in the form of a flexible finite system for topical application.

14. The composition of claim 13, wherein the composition comprises amounts of the phytoestrogen and antioxidant effective to reduce skin irritation associated with use of the system over an application period of at least one day.

15. The composition of claim 14, wherein the composition comprises amounts of the phytoestrogen and antioxidant effective to reduce skin irritation associated with use of the system over an application period of at least three days.

16. The composition of claim 13, wherein the composition comprises amounts of the phytoestrogen and antioxidant effective to reduce skin irritation associated with use of the system over an application period of at least seven days.

17. The composition of claim 13, wherein the phytoestrogen and antioxidant are present in a skin-contacting layer of the system.

18. The composition of claim 13, wherein the phytoestrogen and antioxidant are present in a drug-containing polymer matrix layer of the system.

19. A method of reducing the skin or mucosa irritating effects of a transdermal or transmucosal pharmaceutical composition, comprising formulating a transdermal or transmucosal pharmaceutical composition with a phytoestrogen and an antioxidant in amounts effective to reduce the skin or mucosa irritation potential of the composition.

20. A method of reducing the skin or mucosa irritating effects of transdermal or transmucosal drug delivery, comprising administering to a subject in need of transdermal or transmucosal drug delivery a transdermal or transmucosal pharmaceutical composition comprising a phytoestrogen and an antioxidant in amounts effective to reduce the skin or mucosa irritation potential of the composition.
